(19) United States
(12) Patent Application Publication
(10) Pub. No.: US 2012/0190605 A1
(43) Pub. Date: Jul. 26, 2012

(54) USE OF TALLOW FATTY ALCOHOL ETHOXYLATES IN MACHINE DISHWASHING

(75) Inventors: Sonja Fischer, Plankstadt (DE); Jürgen Tropsch, Roemerberg (DE); Heike Weber, Mannheim (DE); Roland Ettl, Altlussheim (DE)

(73) Assignee: BASF SE, Ludwigshafen (DE)

(21) Appl. No.: 13/354,776

(22) Filed: Jan. 20, 2012

Related U.S. Application Data
(60) Provisional application No. 61/434,837, filed on Jan. 21, 2011.

(51) Int. Cl. C11D 3/60; C07C 43/11; C07C 43/10

(52) U.S. Cl. 510/220; 568/613

(57) ABSTRACT

The invention relates to nonionic surfactants of ethoxylates of linear C₁₆-C₁₈-alcohols with, on average, 8.5 to 9.5 mol of ethylene oxide per mole of linear C₁₆-C₁₈-alcohol.

The invention relates to nonionic surfactants of tallow fatty alcohol ethoxylates, to their use and to dishwashing detergents comprising the nonionic surfactants.

During machine dishwashing, the dishes should be obtained in a residue-free washed state with a faultlessly shiny surface, for which purpose a detergent, a rinse aid and regenerating salt for water softening usually have to be used. The “3 in 1” dishwashing compositions, introduced to the market in 2001, combine the function of detergent, rinse aid and regenerating salt in one product. Besides detergent components for removing the soiling on the dishes, they comprise integrated rinse aid surfactants which ensure water runs off the whole area of the dishes during the rinsing and drying cycle and thus prevent lime marks and watermarks. Additionally, they have components for binding the hardness-forming calcium and magnesium ions. Consequently, there is no need for the consumer to replenish rinse aid and salt in the dishwasher. The incorporation of further functions (e.g. protection against glass corrosion and protection against the tarnishing of silver) led to the development of x in 1 (where e.g. x=6 or 9) or “all in one” products.

WO 2008/132131 discloses the use of a combination of at least one alcohol alkoxylate, at least one short-chain alcohol ethoxylate, at least one polymer containing sulfonate groups and/or at least one hydrophilically modified polycarboxylate and optionally a polycarboxylate, together with generally customary further constituents, for improving the clear-rinse performance in phosphate-containing machine dishwashing detergents even in the event of considerably greater water hardness. WO2008/132131 discloses the use of the same combination for improving the clear-rinse performance in phosphate-free machine dishwashing detergents.

The alcohol alkoxylate has the general formula (I)

R¹—(OCH₂CHR²)ₓ(OCH₂CHR³)_{y}—OR⁴ (I)

in which
R¹ is a linear or branched C₆-C₂₄-alkyl radical,
R², R³ are hydrogen or linear or a branched C₁-C₆-alkyl radical,
R⁴ is hydrogen or a linear or branched C₁-C₈-alkyl radical,
x, y are an average value in the range from 0.5-80,
where the individual alkylene oxide units may be present as a block or in random distribution.

As alcohol alkoxylates of the general formula (I), very particular preference is given to using:
C₁₃- to C₁₅-oxo alcohols+10 units of ethylene oxide+2 units of butylene oxide,
iso-C₁₀-alcohols+10 units of ethylene oxide+1.5 units of pentene oxide,
C₁₀- to C₁₂-fatty alcohols+9 units of ethylene oxide+5 units of propylene oxide,
C₁₃- to C₁₅-oxo alcohols+4.46 units of ethylene oxide+0.86 units of butylene oxide, terminally capped with a methyl group,
2-propylheptanol+6 units of ethylene oxide+4.5 units of propylene oxide or mixtures thereof.

DE 102 33 834 A discloses, as nonionic surfactants in machine dishwashing detergents, alkoxylated, preferably ethoxylated, primary alcohols having 8 to 18 carbon atoms and on average 1 to 12 mol of ethylene oxide (EO) per mole of alcohol. Preference is given to alcohol ethoxylates of alcohols of native origin having 12 to 18 carbon atoms, such as coconut, palm, tallow fatty or oleyl alcohol having on average 2 to 8 mol of EO per mole of alcohol. Specific mention may be made, inter alia, of C₁₂-C₁₄-alcohols with 3 or 4 EO, C₉-C₁₁-alcohols with 7 EO, C₁₃-C₁₅-alcohols with 3, 5, 7 or 8 EO and C₁₂-C₁₈-alcohols with 5 EO.

It is an object of the present invention to improve the clear-rinse performance dishwashing detergents, in particular of so-called 3-in-1 dishwashing detergents, during dishwashing.

It is in particular an object of the present invention to increase the clear-rinse performance of the dishwashing detergents at water hardnesses above 14° German hardness.

The object is achieved by nonionic surfactants of ethoxylates of linear C₁₆-C₁₈-alcohols with, on average, 8.5 to 9.5 mol of ethylene oxide per mole of linear C₁₆-C₁₈-alcohol.

Surprisingly, it has been found that nonionic surfactants based on tallow fatty alcohols (C₁₆-C₁₈-alcohols) with ca. 9 mol of ethylene oxide per mole of alcohol bring about a considerably better clear-rinse performance than those with ca. 7 or ca. 11 mol of ethylene oxide per mole of alcohol.

The nonionic surfactants according to the invention preferably comprise ethoxylates of linear C₁₆-C₁₈-alcohols of the formula (I)

R¹—(OCH₂CH₂)ₓ—OR² (I)

where
R¹ is a linear C₁₆-C₁₈-alkyl radical,
R² is hydrogen or a linear or branched C₁-C₆-alkyl radical,
x is 8, 9 or 10.

The nonionic surfactants according to the invention particularly preferably comprise at least 80% by weight of ethoxylates of linear C₁₆-C₁₈-alcohols of the formula (I) where x=8, 9 or 10 ethylene oxide units.

In particular, the nonionic surfactants comprise at least 80% by weight of ethoxylates of linear C₁₆-C₁₈-alcohols of the formula (I) where x=9 ethylene oxide units.

Preferred nonionic surfactants furthermore comprise, on average, 8.8 to 9.2 mol of ethylene oxide, in particular 8.9 to 9.1 mol of ethylene oxide, per mole of linear C₁₆-C₁₈-alcohol.

Preferred linear C₁₆-C₁₈-alcohols are n-hexadecanol and n-octadecanol. These can be obtained by hydrogenation of natural tallow fat. Mixtures of linear C₁₆- and C₁₈-alcohols of natural origin are also known as tallow fatty alcohol. Tallow fatty alcohols have to a small extent unsaturated fractions, in particular fractions of mono- or polyunsaturated C₁₆- and C₁₈-alcohols. The iodine number of the tallow fatty alcohols is generally <5 g of I₂/100 g of alcohol, preferably <1 g of I₂/100 g of alcohol.

The compounds of the general formula (I) according to the invention are obtained by alkoxylation of the C₁₆-C₁₈-alcohols with alkylene oxides. Here, if the radical R² is not hydrogen, the alkoxylation can be followed by an etherification, e.g. with dimethyl sulfate.

Preferably, R² is hydrogen or methyl.

The ethoxylation can be carried out, for example, using alkaline catalysts such as alkali metal hydroxides or alkali metal alcoholates. By using these catalysts, specific properties, in particular the homolog distribution of the alkylene oxides, result.

Moreover, the ethoxylation can be carried out using Lewis-acidic catalysts, especially in the presence of BF₃×H₃PO₄, BF₃×dietherate, BF₃, SbCl₆, SnCl₄×2H₂O or hydrotalcite.

The ethoxylation is preferably catalyzed by strong bases, which are expediently added in the form of an alkali metal hydroxide or alkaline earth metal hydroxide, generally in an amount of from 0.1 to 1% by weight, based on the amount of the alcohol R¹—OH.

The alkoxylation can also be carried out with double-metal cyanide catalysts. DMC compounds suitable as catalyst are described, for example, in WO 99/16775 and in DE-A-101 17 273.

The addition reaction is carried out at temperatures of from about 90 to about 240° C., preferably from 120 to 180° C., in a closed vessel. Ethylene oxide is fed to the mixture of C₁₆-C₁₈-alcohol or C₁₆-C₁₈-alcohol mixture and alkali under the vapor pressure of the alkylene oxide mixture prevailing at the selected reaction temperature. If desired, the alkylene oxide can be diluted with up to about 30 to 60 Vol.-% of an inert gas. By doing so, an explosion-like polyaddition or decomposition of the alkylene oxide can be prevented.

The invention also provides the use of the nonionic surfactants according to the invention comprising ethoxylates of linear C₁₆-C₁₈-alcohols with, on average, 8.5 to 9.5 mol of ethylene oxide per mole of alcohol in detergent formulations for machine dishwashing.

The invention further provides a detergent formulation for machine dishwashing comprising, as components:
a) 0.1 to 20% by weight of nonionic surfactants of ethoxylates of linear C₁₆-C₁₈-alcohols with, on average, 8.5 to 9.5 mol of ethylene oxide per mole of C₁₆-C₁₈-alcohol,
b) 0 to 10% by weight of further nonionic surfactants,
c) 0 to 20% by weight of polycarboxylates different from component c),
d) 0 to 50% by weight of complexing agents,
e) 0 to 70% by weight of phosphates,
f) 0 to 60% by weight of further builders and cobuilders,
g) 0 to 30% by weight of bleaches and optionally bleach activators and bleach catalysts,
h) 0 to 8% by weight of enzymes,
i) 0 to 50% by weight of one or more further additives, such as anionic or zwitterionic surfactants, alkali carriers, corrosion inhibitors, antifoams, dyes, fragrances, fillers, organic solvents, tableting auxiliaries, disintegrants, thickeners, solubility promoters and water,
   where the sum of the components a) to i) is 100% by weight.

The detergent formulations according to the invention preferably comprise 0.5 to 15% by weight, particularly preferably 1 to 10% by weight, of the nonionic surfactants a).

Besides the nonionic surfactants of component a) according to the invention, the detergent formulations can comprise up to 10% by weight of further nonionic surfactants different from component a), usually weak- or low-foaming nonionic surfactants. If these are present, they are present in amounts of from 0.1 to 10% by weight, preferably from 0.25 to 5% by weight.

Suitable further nonionic surfactants comprise the surfactants of the general formula (II)

R⁴—O—(CH₂CH₂O)ₚ—(CHR³CH₂O)ₘ—R⁵ (II)

in which R⁴ is a linear or branched alkyl radical having 8 to 22 carbon atoms,
R³ and R⁵, independently of one another, are hydrogen or a linear or branched alkyl radical with 1-10 carbon atoms or H, where R³ is preferably methyl,
p and m, independently of one another, are 0 to 300. Preferably, p=1-100 and m=0-30.

The surfactants of the formula (III) may either be random copolymers or block copolymers, they are preferably block copolymers.

Furthermore, di- and multiblock copolymers formed from ethylene oxide and propylene oxide can be used; these are commercially available, for example, under the name Pluronic® (BASF SE) or Tetronic® (BASF Corporation). Furthermore, reaction products of sorbitan esters with ethylene oxide and/or propylene oxide can be used. Amine oxides or alkyl glycosides are likewise suitable. An overview of suitable further nonionic surfactants is given in EP-A 851 023 and DE-A 198 19 187.

Mixtures of two or more different nonionic surfactants may also be present.

As component c), the detergent formulations according to the invention can comprise 0 to 20% by weight of one or more different polycarboxylates. These can be hydrophilically or hydrophobically modified. If these are present, they are generally present in amounts of from 0.1 to 20% by weight.

Alkali metal salts of homo- and copolymers of acrylic acid or of methacrylic acid are suitable. Of suitability for the copolymerization are monoethylenically unsaturated dicarboxylic acids such as maleic acid, fumaric acid, maleic anhydride, itaconic acid and citraconic acid. A suitable polymer is in particular polyacrylic acid, which preferably has a molar mass of from 2000 to 40,000 g/mol. On account of its superior solubility, from this group, the short-chain polyacrylic acid, which has molar masses of from 2000 to 10,000 g/mol, in particular 3000 to 8000 g/mol, may be preferred. Also of suitability are copolymeric polycarboxylates, in particular those of acrylic acid with methacrylic acid and of acrylic acid or methacrylic acid with maleic acid and/or fumaric acid.

It is also possible to use copolymers of at least one monomer from the group consisting of monoethylenically unsaturated C₃-C₁₀-mono- or dicarboxylic acids or anhydrides thereof, such as maleic acid, maleic anhydride, acrylic acid, methacrylic acid, fumaric acid, itaconic acid and citraconic acid with at least one hydrophilically or hydrophobically modified monomer.

Suitable hydrophobic monomers are, for example, isobutene, diisobutene, butene, pentene, hexene and styrene, olefins having 10 or more carbon atoms or mixtures thereof, such as, for example, 1-decene, 1-dodecene, 1-tetradecene, 1-hexadecene, 1-octadecene, 1-eicosene, 1-docosene, 1-tetracosene and 1-hexacosene, C₂₂-alpha-olefin, a mixture of C₂₀-C₂₄-alpha-olefins and polyisobutene with, on average, 12 to 100 carbon atoms.

Suitable hydrophilic monomers are monomers with sulfonate or phosphonate groups, and also nonionic monomers with hydroxy functions or alkylene oxide groups. For example, mention may be made of: allyl alcohol, isoprenol, methoxypolyethylene glycol (meth)acrylate, methoxypolypropylene glycol (meth)acrylate, methoxypolybutylene glycol (meth)acrylate, methoxypoly(propylene oxide-co-ethylene oxide) (meth)acrylate, ethoxypolyethylene glycol (meth)acrylate, ethoxypolypropylene glycol (meth)acrylate, ethoxypolybutylene glycol (meth)acrylate and ethoxypoly(propylene oxide-co-ethylene oxide) (meth)acrylate. The polyalkylene glycols here comprise 3 to 50, in particular 5 to 40 and especially 10 to 30, alkylene oxide units.

Particularly preferred monomers containing sulfonic acid groups here are 1-acrylamido-1-propanesulfonic acid, 2-acrylamido-2-propanesulfonic acid, 2-acrylamido-2-methyl-propanesulfonic acid, 2-methacrylamido-2-methylpropanesulfonic acid, 3-methacrylamido-2-hydroxypropanesulfonic acid, allylsulfonic acid, methallylsulfonic acid, allyloxy-benzenesulfonic acid, methallyloxybenzenesulfonic acid, 2-hydroxy-3-(2-propenyloxy)-propanesulfonic acid, 2-methyl-2-propene-1-sulfonic acid, styrenesulfonic acid, vinyl-sulfonic acid, 3-sulfopropyl acrylate, 2-sulfoethyl methacrylate, 3-sulfopropyl methacrylate, sulfomethacrylamide, sulfomethylmethacrylamide, and salts of the specified acids, such as their sodium, potassium or ammonium salts.

Particularly preferred monomers containing phosphonate groups are vinylphosphonic acid and its salts.

Moreover, it is also possible to additionally use amphoteric and cationic polymers.

As component d), the detergent formulations according to the invention can comprise 0 to 50% by weight of one or more complexing agents. If complexing agents are present, these are present in amounts of from 0.1 to 50% by weight, preferably 1 to 45% by weight and particularly preferably 1 to 40% by weight. Preferred complexing agents are selected from the group consisting of nitrilotriacetic acid, ethylenediaminetetraacetic acid, diethylene-triaminepentaacetic acid, hydroxyethylethylenediaminetriacetic acid and methylglycine-diacetic acid, glutamic acid diacetic acid, iminodisuccinic acid, hydroxyiminodisuccinic acid, ethylenediaminedisuccinic acid, aspartic acid diacetic acid, and salts thereof. Particularly preferred complexing agents e) are methylglycinediacetic acid and salts thereof.

As component e), the detergents according to the invention can comprise 0 to 70% by weight of phosphates. If the detergent comprises phosphates, it generally comprises these in amounts of from 1 to 70% by weight, preferably from 5 to 60% by weight, particularly preferably from 20 to 55% by weight.

Among the multitude of commercially available phosphates, the alkali metal phosphates, with particular preference pentasodium or pentapotassium triphosphate (sodium or potassium tripolyphosphate) have the greatest importance in the detergents and cleaners industry.

Suitable phosphates for dishwashing detergents are in particular alkali metal phosphates and polymeric alkali metal phosphates, which may be present in the form of their alkaline, neutral or acidic sodium or potassium salts. Examples of such phosphates are trisodium phosphate, tetrasodium diphosphate, disodium dihydrogendiphosphate, pentasodium tripolyphosphate, so-called sodium hexametaphosphate, oligomeric trisodium phosphate with a degree of oligomerization of from 5 to 1000, preferably 5 to 50, and the corresponding potassium salts, or mixtures of sodium hexametaphosphate and the corresponding potassium salts, or mixtures of the sodium and potassium salts. Particular preference is given to the tripolyphosphate salts.

As component f), the detergents according to the invention can comprise 0 to 60% by weight of builders and cobuilders. If the detergent comprises builders and cobuilders, it comprises these generally in amounts of from 0.1 to 60% by weight. Builders and cobuilders are water-soluble or water-insoluble substances whose main task consists in the binding of calcium and magnesium ions.

These may be low molecular weight carboxylic acids and salts thereof, such as alkali metal citrates, in particular anhydrous trisodium citrate or trisodium citrate dihydrate, alkali metal succinates, alkali metal malonates, fatty acid sulfonates, oxydisuccinate, alkyl or alkenyl disuccinates, gluconic acids, oxadiacetates, carboxymethyloxysuccinates, tartrate mono-succinate, tartrate disuccinate, tartrate monoacetate, tartrate diacetate and α-hydroxy-propionic acid.

A further substance class with cobuilder properties which may be present in the detergents according to the invention are the phosphonates. These are in particular hydroxyalkane- or aminoalkanephosphonates. Among the hydroxyalkanephosphonates, 1-hydroxyethane-1,1-diphosphonate (HEDP) is of particular importance as a cobuilder. It is preferably used as the sodium salt, with the disodium salt giving a neutral reaction and the tetrasodium salt giving an alkaline reaction (pH 9). Suitable aminoalkanephosphonates are preferably ethylenediaminetetramethylenephosphonate (EDTMP), diethylenetriaminepentamethylene-phosphonate (DTPMP) and their higher homologs. They are preferably used in the form of the neutrally reacting sodium salts, e.g. as hexasodium salt of EDTMP or as hepta- and octa-sodium salt of DTPMP. From the class of phosphonates, the builder used here is preferably HEDP. Moreover, the aminoalkanephosphonates have a marked capacity for binding heavy metals. Accordingly, it may be preferred, particularly if the compositions also comprise bleaches, to use aminoalkanephosphonates, in particular DTPMP, or to use mixtures of the stated phosphonates.

A further substance class in the builder system is the silicates. Crystalline layered silicates with the general formula NaMSiₓO₂ₓ₊₁.yH₂O, where M is sodium or hydrogen, x is a number from 1.9 to 22, preferably from 1.9 to 4, where particularly preferred values for x are 2, 3 or 4 and y is a number from 0 to 33, preferably 0 to 20, may be present. In addition, amorphous sodium silicates with an SiO₂:Na₂O ratio of 1:3.5, preferably of 1.6:3 and in particular of 2:2.8, can be used.

Furthermore, carbonates and hydrogencarbonates are used, of which the alkali metal salts, in particular sodium salts, are preferred.

As component g), the detergent formulations according to the invention can comprise 0 to 30% by weight of bleaches, optionally bleach activators and optionally bleach catalysts. If the detergent formulations comprise bleaches, bleach activators or bleach catalysts, they comprise these in amounts of in total 0.1 to 30% by weight, preferably 1 to 30% by weight and particularly preferably 5 to 30% by weight.

Bleaches are divided into oxygen bleaches and chlorine-containing bleaches. Alkali metal perborates and hydrates thereof and also alkali metal percarbonates are used as oxygen bleaches. Preferred bleaches here are sodium perborate in the form of the mono- or tetrahydrate, sodium percarbonate or the hydrates of sodium percarbonate.

Persulfates and hydrogen peroxide can likewise be used as oxygen bleaches.

Typical oxygen bleaches are also organic peracids, such as, for example, perbenzoic acid, peroxy-alpha-naphthoic acid, peroxylauric acid, peroxystearic acid, phthalimidoperoxy-caproic acid, 1,12-diperoxydodecanedioic acid, 1,9-diperoxyazelaic acid, diperoxoiso-phthalic acid or 2-decyldiperoxybutane-1,4-dioic acid.

Moreover, the following oxygen bleaches can also be used in the detergent formulation:

Cationic peroxy acids, which are described in U.S. Pat. No. 5,422,028, U.S. Pat. No. 5,294,362 and U.S. Pat. No. 5,292,447, and sulfonylperoxy acids, which are described in U.S. Pat. No. 5,039,447.

Chlorine-containing bleaches and also the combination of chlorine-containing bleaches with peroxide-containing bleaches can likewise be used. Known chlorine-containing bleaches are, for example, 1,3-dichloro-5,5-dimethylhydantoin, N-chlorosulfamide, chloramine T, dichloramine T, chloramine B, N,N′-dichlorobenzoylurea, p-toluenesulfondichloroamide or trichloroethylamine. Preferred chlorine-containing bleaches are sodium hypochlorite, calcium hypochlorite, potassium hypochlorite, magnesium hypochlorite, potassium dichloroisocyanurate or sodium dichloroisocyanurate.

Chlorine-containing bleaches are used in amounts of in general 0.1 to 20% by weight, preferably from 0.2 to 10% by weight, particularly preferably from 0.3 to 8% by weight, based on the total detergent formulation.

Furthermore, bleach stabilizers such as, for example, phosphonates, borates, metaborates, metasilicates or magnesium salts may be added in small amounts.

Bleach activators are compounds which, under perhydrolysis conditions, produce aliphatic peroxocarboxylic acids having preferably 1 to 10 carbon atoms, in particular 2 to 4 carbon atoms, and/or substituted perbenzoic acid. Compounds which comprise one or more N- and/or O-acyl groups and/or optionally substituted benzoyl groups are suitable, for example substances from the class of the anhydrides, esters, imides, acylated imidazoles or oximes. Examples are tetraacetylethylenediamine (TAED), tetraacetylmethylenediamine (TAMD), tetraacetylglycoluril (TAGU), tetraacetylhexylenediamine (TAHD), N-acylimides, such as, for example, N-nonanoylsuccinimide (NOSI), acylated phenolsulfonates, such as, for example, n-nonanoyl- or isononanoyloxybenzenesulfonates (n- or iso-NOBS), pentaacetyl-glucose (PAG), 1,5-diacetyl-2,2-dioxohexahydro-1,3,5-triazine (DADHT) or isatoic anhydride (ISA). Likewise suitable as bleach activators are nitrile quats, such as, for example, N-methylmorpholinium acetonitrile salts (MMA salts) or trimethylammonium acetonitrile salts (TMAQ salts).

Preferably suitable are bleach activators from the group consisting of polyacylated alkylenediamines, particularly preferably TAED, N-acylimides, particularly preferably NOSI, acylated phenolsulfonates, particularly preferably n- or iso-NOBS, MMA and TMAQ.

Bleach activators are used in amounts of in general 0.1 to 10% by weight, preferably from 1 to 9% by weight, particularly preferably from 1.5 to 8% by weight, based on the total detergent formulation.

In addition to the conventional bleach activators, or instead of them, it is also possible for so-called bleach catalysts to be present. These substances are bleach-boosting transition metal salts or transition metal complexes, such as, for example, manganese-, iron-, cobalt-, ruthenium- or molybdenum-salen complexes or carbonyl complexes. Manganese, iron, cobalt, ruthenium, molybdenum, titanium, vanadium and copper complexes with nitrogen-containing tripod ligands, and also cobalt-, iron-, copper- and ruthenium-amine complexes can also be used as bleach catalysts.

As component h), the detergent formulations according to the invention can comprise 0 to 8% by weight of enzymes. If the detergent formulations comprise enzymes, they generally comprise these in amounts of from 0.1 to 8% by weight. Enzymes can be added to the detergent in order to increase the performance of the detergents or to ensure detergency of equal quality under milder conditions. The enzymes used most often include lipases, amylases, cellulases and proteases. Furthermore, esterases, pectinases, lactases and peroxidases, for example, can also be used.

Furthermore, the detergents according to the invention can comprise, as component i), 0 to 50% by weight of one or more further additives, such as anionic or zwitterionic surfactants, alkali carriers, corrosion inhibitors, antifoams, dyes, fragrances, fillers, organic solvents, tableting auxiliaries, disintegrants, thickeners, solubility promoters and water. If the detergent formulation comprises further additives, these are generally present in amounts of from 0.1 to 50% by weight.

The formulations can comprise anionic or zwitterionic surfactants, preferably in a mixture with nonionic surfactants. Suitable anionic and zwitterionic surfactants are given in EP-A 851 023 and DE-A 198 19 187.

Alkali carriers may be present as further constituents of the detergent formulation. Besides the ammonium or alkali metal carbonates, ammonium or alkali metal hydrogencarbonates and ammonium or alkali metal sesquicarbonates already mentioned under the builder substances, alkali carriers which can be used are also ammonium or alkali metal hydroxides, ammonium or alkali metal silicates and ammonium or alkali metal meta-silicates, and also mixtures of the aforementioned substances.

Corrosion inhibitors which can be used are silver protectants from the group of the triazoles, the benzotriazoles, the bisbenzotriazoles, the aminotriazoles, the alkylamino-triazoles and the transition metal salts or complexes.

To prevent glass corrosion, which becomes noticeable from clouding, iridescence, streaks and lines on the glasses, glass corrosion inhibitors are used. Preferred glass corrosion inhibitors are from the group of the magnesium, zinc and bismuth salts and complexes.

Paraffin oils and silicone oils can optionally be used as antifoam and for protecting plastic and metal surfaces. Antifoams are generally used in fractions of from 0.001% by weight to 5% by weight. Moreover, dyes such as, for example, Patent blue, preservatives such as, for example, Kathon CG, perfumes and other fragrances can be added to the detergent formulation according to the invention.

A suitable filler is, for example, sodium sulfate.

The detergent formulations according to the invention can be provided in packaged form, in a liquid or solid form, with one or more phases, as tablets or in the form of other dosing units, or in unpackaged form.

The invention is illustrated in more detail by the examples below.

### EXAMPLES

The nonionic surfactants T 9, T 7 and T 11 were tested in the following phosphate-free formulations PF1 and also in the phosphate-based formulation P1.

T 9: tallow fatty alcohol ethoxylate with, on average, 9 mol of ethylene oxide per mole of alcohol;

T 11: tallow fatty alcohol ethoxylate with, on average, 11 mol of ethylene oxide per mole of alcohol;

T 7: tallow fatty alcohol ethoxylate with, on average, 7 mol of ethylene oxide per mole of alcohol.

The composition of the formulations is given in table 1 (data in % by weight).

TABLE 1

| | PF 1 | P1 |
|---|---|---|
| Protease | 1 | 1 |
| Amylase | 0.2 | 0.2 |
| Nonionic surfactant | 5 | 3 |
| Acrylic acid/AMPS-copolymer | 10 | 6.5 |
| (Sokalan ® CP50) | | |
| Sodium percarbonate | 10.5 | 14 |
| Tetraacetylethylenediamine | 4 | 4 |
| Sodium disilicate | 2 | 2 |
| Sodium tripolyphosphate | — | 50 |
| Solvay soda heavy | 18.8 | 18.8 |
| Sodium citrate dihydrate | 33 | — |
| Methylglycinediacetic acid | 15 | — |
| Hydroxyethane-(1,1-diphosphonic | 0.5 | 0.5 |
| acid) | | |

The following experimental conditions were observed here:
Dishwasher: Miele G 1222 SCL
Program: 50° C. with R-time 2 [8 min] (without prewash)
Dishes: 3 knives (WMF Tafelmesser Berlin, Monoblock)
   3 glasses Amsterdam 0.21
   3 BREAKFAST PLATES “OCEAN BLUE” (MELAMINE)
   3 porcelain PLATES WITH RIMS FLAT 19 cm
Arrangement: Knives in the cutlery drawer, glasses in the upper basket, plates in the lower basket
Dishwashing detergent: 21 g
Addition of soiling: 100 g of clear-rinse soiling (comprises egg, starch and grease), is metered in frozen form
Rinse temperature: 65° C.
Water hardness: 21° German hardness (Ca/Mg):HCO3 (3:1):1.35
Wash cycles: 6; break in between for 1 h in each case (10 min with door open, 50 min with door closed)
Evaluation: Visually after 6 wash cycles

The evaluation of the dishes was carried out after 6 cycles in a darkened chamber under light behind an apertured diaphragm using a grading scale from 10 (very good) to 1 (very poor). Grades from 1-10 for spotting (very many, intensive spots=1 to no spots=10) and also for filming, the grades 1-10 (1=very severe filming, 10=no filming) were awarded.

The test results are summarized in table 2.

TABLE 2

| | Spotting | | | | Filming | | | |
|---|---|---|---|---|---|---|---|---|
| | Knives | Glasses | Melamine | Porcelain | Knives | Glasses | Melamine | Porcelain |
| T 9 | 10 | 7 | 7 | 9 | 7 | 6 | 7 | 8 |
| T 11 | 6 | 1 | 3 | 2 | 6 | 8 | 8 | 9 |
| T 7 | 8 | 3 | 2 | 3 | 7 | 9 | 7 | 9 |

As can be seen in the table, the surfactant T9 is considerably better as regards spotting than the comparative surfactants T7 and T11. In the case of filming, comparably good results are obtained with all 3 surfactants.

1. A nonionic surfactant of ethoxylates of linear C₁₆-C₁₈-alcohols with, on average, 8.5 to 9.5 mol of ethylene oxide per mole of linear C₁₆-C₁₈-alcohol.

2. The nonionic surfactant according to claim 1, comprising ethoxylates of linear C₁₆-C₁₈-alcohols of the formula (I)
R¹—(OCH₂CH₂)ₓ—OR² (I)
where
R¹ is a linear C₁₆-C₁₈-alkyl radical,
R² is hydrogen or a linear or branched C₁-C₆-alkyl radical,
x is 8, 9 or 10.

3. The nonionic surfactant according to claim 2, comprising at least 80% by weight of ethoxylates of linear C₁₆-C₁₈-alcohols of the formula (I) where x=8, 9 or 10 ethylene oxide units.

4. The nonionic surfactant according to claim 3, comprising at least 80% by weight of ethoxylates of linear C₁₆-C₁₈-alcohols of the formula (I) where x=9 ethylene oxide units.

5. The nonionic surfactant according claim 1 comprising, on average, 8.8-9.2 mol of ethylene oxide per mole of linear C₁₆-C₁₈-alcohol.

6. A detergent formulation for machine dishwashing, comprising
a) 0.1 to 20% by weight of nonionic surfactants of ethoxylates of linear C₁₆-C₁₈-alcohols with, on average, 8.5 to 9.5 mol of ethylene oxide per mole of linear C₁₆-C₁₈-alcohol according to any one of claims 1 to 5,
b) 0 to 10% by weight of further nonionic surfactants,
c) 0 to 20% by weight of polycarboxylates,
d) 0 to 50% by weight of complexing agents,
e) 0 to 70% by weight of phosphates,
f) 0 to 60% by weight of further builders and cobuilders,
g) 0 to 30% by weight of bleaches and optionally bleach activators and bleach catalysts,
h) 0 to 8% by weight of enzymes,
i) 0 to 50% by weight of one or more further additives such as anionic or zwitterionic surfactants, alkali carriers, corrosion inhibitors, antifoams, dyes, fragrances, fillers, organic solvents, tableting auxiliaries, disintegrants, thickeners, solubility promoters and water,
where the sum of the components a) to i) is 100% by weight.

7. The detergent formulation of claim 6 comprising, as component a) ethoxylates of linear C₁₆-C₁₈-alcohols of the formula (I)
R¹—(OCH₂CH₂)ₓ—OR² (I)
where
R¹ is a linear C₁₆-C₁₈-alkyl radical,
R² is hydrogen or a linear or branched C₁-C₆-alkyl radical,
x is 8, 9 or 10.

8. The detergent formulation of claim 7, wherein component a) comprises least 80% by weight of ethoxylates of linear C₁₆-C₁₈-alcohols of the formula (I) where x=8, 9 or 10 ethylene oxide units.

9. The detergent formulation of claim 8, wherein component a) comprises at least 80% by weight of ethoxylates of linear C₁₆-C₁₈-alcohols of the formula (I) where x=9 ethylene oxide units.

10. The detergent formulation of claim 7, wherein component a) comprises, on average, 8.8-9.2 mol of ethylene oxide per mole of linear C₁₆-C₁₈-alcohol.
